(19) United States
(12) Patent Application Publication
(10) Pub. No.: US 2002/0165468 A1
(43) Pub. Date: Nov. 7, 2002

(54) Combined catheter and input device

(75) Inventors: David Tolkowsky, Tel Aviv (IL); Pinhas Gilboa, Haifa (IL)

Correspondence Address: DR. MARK FRIEDMAN, c/o BILL POLKINGHORN-DISCOVERY DISPATCH, 9003 FORIN WAY, UPPER MARLBORO, MD, 20772, US

(73) Assignee: Super Dimension Ltd.

(21) Appl. No.: 09/845,202

(22) Filed: May 1, 2001

(51) Int. Cl. A61B 5/103; A61B 5/117

(52) U.S. Cl. 600/587

(57) ABSTRACT

A combined catheter and input device for use by a medical practitioner to designate points of interest reached by the device includes a catheter and a location sensing system. The location sensing system, which includes at least one element associated with a distal portion of the catheter, determines a position of at least one point on the distal portion. An input device is mounted at the proximal portion and configured to be manually actuated to designate a current position of the at least one point as a point of interest.

### FIELD AND BACKGROUND OF THE INVENTION

The present invention relates to catheters and, in particular, it concerns a combined catheter and input device.

Co-assigned PCT Publication No. WO00/16684 discloses a technique referred to herein as the Point Of Interest (POI) method, used for marking a location of a medical tool inside the body for later use. Co-assigned PCT Publication No. WO00/10456 discloses a method and an apparatus for tracking a medical tool inside the body are described. U.S. Pat. No. 5,738,096 “Cardiac Electrodynamics” and the associated system commercially available under the trademark “CARTO” from Biosense, Israel, describes a system for mapping the functionality and geometry of an internal organ. This is done by measuring the location of a probe and measuring a local biophysical value. Acquiring many data points allows drawing a map of the said biophysical values. Activation of the measurement process is done either by the practitioner's assistant using a computer input device such as a keyboard or a mouse, or by the practitioner using a foot switch. All of the aforementioned publications are hereby incorporated by reference.

Catheters for the application of medical treatment inside the body are well-known. For example, steerable catheters are commonly used for treatment inside a cavity of the heart. One known treatment is to deliver energy, usually RF energy, to ablate a portion of the myocardium for the purpose of curing cardiac arrhythmia. Another known use of a catheter is, while equipped with an ultrasound probe, to produce an intracardiac image.

FIG. 1A illustrates a typical steerable catheter. The catheter has a long tube 104, typically of 1 to 3 mm diameter, for insertion along the blood vessels, a handle 100 and a wire 106 for connecting the catheter to the external equipment. A lever or other actuator 102 manipulates the end portion 110 of the catheter. When pulled, as shown in FIG. 1B, end portion 110 of the catheter bends, allowing the medical practitioner to steer the catheter inside the cavities of the heart.

Activation of the measurement of a POI and simultaneously the marking of such on the computer's display as described in WO 00/16684, or measuring local and location information as described in U.S. Pat. No. 5,738,096, is performed by holding the catheter (having an element of a location sensing system incorporated into its tip) at the location to be logged, at which time the system is triggered to measure and store the information. Triggering is normally performed using a standard computer input device such as a keyboard or computer mouse. Since the medical practitioner is required to keep his hand sterile, the computer peripherals are necessarily operated by the practitioner's assistant. This division of POI designation between two people may be inconvenient and can lead to difficulties in cooperation and coordination between the practitioner and his or her assistant.

An alternative technique for triggering designation of a POI is by use of a foot switch. The use of a foot switch avoids the problem of sterility of the practitioner's hands, thereby allowing the practitioner to designate the P01 directly himself. Nevertheless, the use of a foot switch poses its own particular problems. Specifically, similar foot switches are also widely used for controlling multiple devices in an operating theatre, in this case typically including for triggering a fluoroscope imaging device, and to operate the RF generator. Since a separate foot switch must be provided for each such device, the practitioner is often faced with multiple foot switches with an accompanying risk the practitioner could get confused between the switches.

There is therefore a need for a device which would enable a practitioner to designate a point of interest himself whenever desired without confusion between multiple input devices, and without violating sterility, while avoiding the need to be closely coordinated with the assistant.

### SUMMARY OF THE INVENTION

The present invention is a combined catheter and input device.

According to the teachings of the present invention there is provided, a combined catheter and input device for use by a medical practitioner to designate points of interest reached by the device within the body of a patient, the device comprising: (a) a catheter having a distal portion for insertion into the body and a proximal portion configured to be held by the practitioner; (b) a location sensing system including at least one element associated with the distal portion of the catheter, the location sensing system determining a position of at least one point on the distal portion; and (c) an input device mounted at the proximal portion and configured to be manually actuated to designate a current position of the at least one point as a point of interest.

According to a further feature of the present invention, the catheter is a steerable catheter further including a steering mechanism manually operable from the proximal portion so as to steer the distal portion.

### BRIEF DESCRIPTION OF THE DRAWINGS

The invention is herein described, by way of example only, with reference to the accompanying drawings, wherein:

FIGS. 1A and 1B are schematic representations of a steerable catheter in a non-deflected and deflected state, respectively;

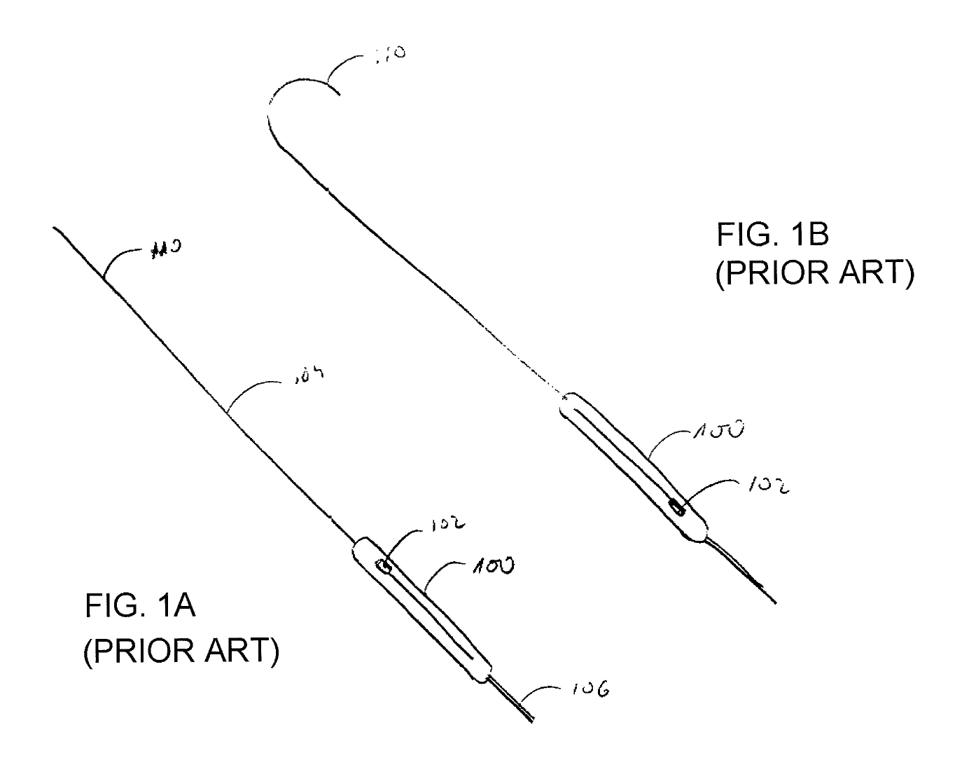

FIG. 2 is a schematic representation of a system employing a combined catheter and input device, constructed and operative according to the teachings of the present invention;

FIG. 3 is a schematic enlarged isometric view of a proximal portion of a first implementation of the combined catheter and input device of FIG. 2; and

FIG. 4 is a schematic enlarged isometric view of a proximal portion of a second implementation of the combined catheter and input device of FIG. 2.

### DESCRIPTION OF THE PREFERRED EMBODIMENTS

The present invention is a combined catheter and input device.

The principles and operation of devices according to the present invention may be better understood with reference to the drawings and the accompanying description.

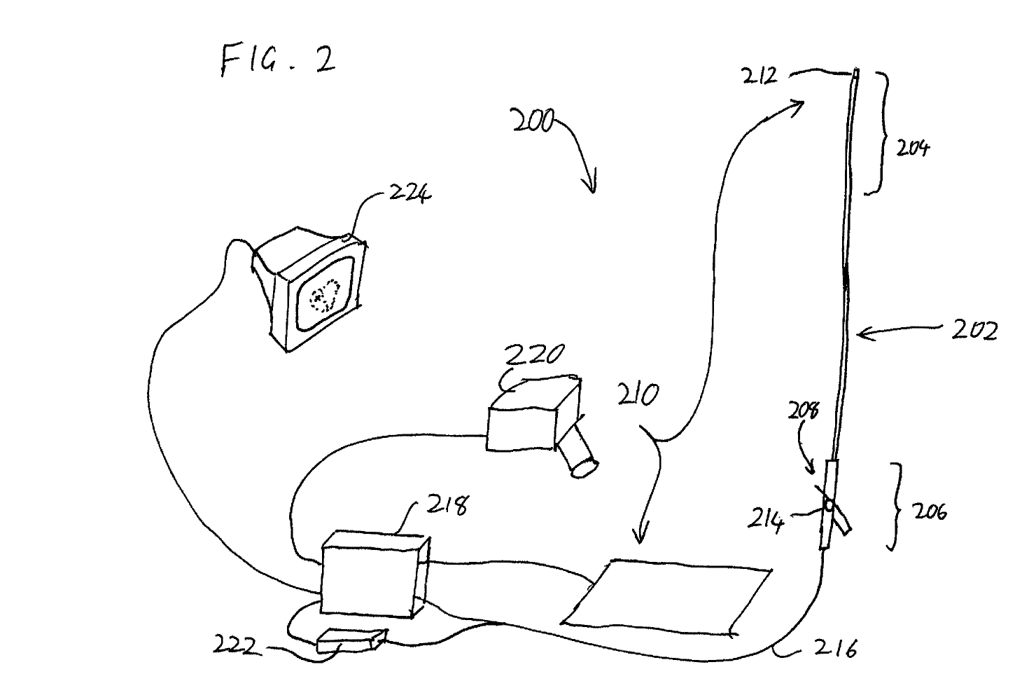

Referring now to the drawings, FIGS. 2-4 show parts of a preferred implementation of a system, generally designated 200, employing a combined catheter and input device for use by a medical practitioner to designate points of interest reached by the device. Generally speaking, system 200 includes a catheter 202 having a distal portion 204 for insertion into the patient's body and a proximal portion 206 configured as a handle to be held by the practitioner. In the preferred implementation shown here, catheter 202 is implemented as a steerable catheter which includes a steering mechanism 208 manually operable from proximal portion 206 so as to steer the distal portion. The device also includes a location sensing system 210 including at least one element 212 associated with distal portion 204. Location sensing system 210 determines a position of at least one point on distal portion 204, typically corresponding to the position of element 212. Details of a suitable location sensing system may, by way of example, be found in the publications incorporated by reference above.

Parenthetically, it should be noted that element 212 is not necessarily a dedicated, structurally distinct element and may, in certain cases, be combined with another element of catheter 202. By way of example, U.S. Pat. No. 5,697,377 describes a location sensing system in which the ablation electrode of a catheter can also serve as a sensor electrode for the location system. Nevertheless, such an implementation is thought to be inferior to an implementation using a dedicated sensor such as that of the system described in the aforementioned PCT Publication No. WO00/10456.

It is a particular feature of the present invention that catheter 202 also includes an input device 214 mounted at proximal portion 206 and configured to be manually actuated to designate a current position of the at least one point as a point of interest (POI). The presence of input device 214 converts catheter 202 into a highly convenient pointing and input device for designating points of interest. As a result, the practitioner can mark a point of interest: (i) whenever desired, (ii) in a very convenient manner as his/her hands are already holding the catheter, (iii) without any potential confusion among multiple input devices, (iv) without violating sterility and (v) with no need to be closely coordinated with the assistant.

Structurally, input device 214 is typically linked by one or more electrical lead 216 to a computer 218 which is associated with location sensing system 210. Other elements of system 200 such as a fluoroscope 220 and an RF ablation device controller 222 are preferably also integrated with computer 218 and, in the case of the fluoroscope, with location sensing system 210, to allow information from the multiple devices to be combined for display on a display screen 224.

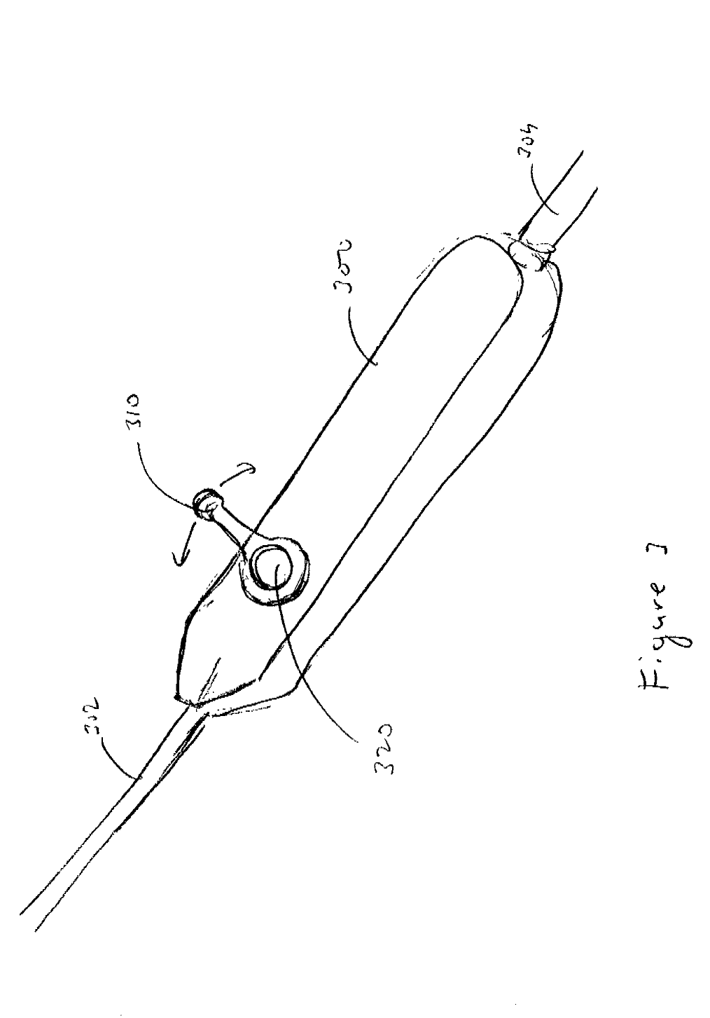

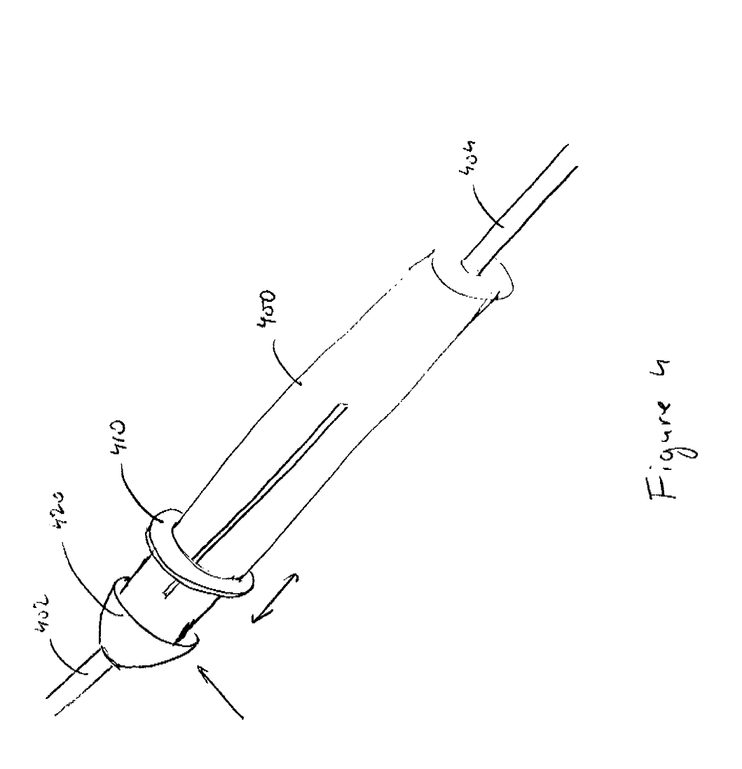

Input device 214 is typically implemented as a simple electric switch mounted somewhere in or on the handle formed in proximal portion 206. By way of non-limiting examples, two possible deployments of the switch will now be illustrated with reference to FIGS. 3 and 4.

FIG. 3 shows one implementation for combining a switch into a handle 300 formed in the proximal portion of a steerable catheter. In this example, the catheter is steered by rotating a lever 310 which causes a deflection torque by a wire mechanism (not shown). A switch, preferably a flat layer such as a ‘bubble switch’, is attached to the handle in an ergonomic manner, for example at point 320 in the middle of the lever 310.

A second example of an implementation for adding a POI designating switch to a steerable catheter handle is shown in FIG. 4. In this example, handle 400 has a tubular shape. The steering lever is a ring 410 which slides along the handle, producing a deflection torque to be transferred through the catheter tube 402 to its deflectable end portion. The switch is attached to the handle at the proximal end under a cap 420. Pushing the cap from any direction causes the cap to rotate, and closes the contacts placed at the root of the cap.

It should be noted that the principles of the present invention may additionally, or alternatively, be applied to a wide range of other input options and corresponding applications. For example, one or more button may be provided for actuating other devices such as, for example, a fluoroscope. In a more sophisticated implementation (not shown), additional input mechanisms, such as arrow keys or a pointing device (a mini-joystick etc.), may be provided for selecting functions from a menu, typically displayed separately on a nearby display screen, thereby facilitating control of multiple functions or devices directly by the practitioner without requiring him or her to release the catheter.

Although the preferred implementation illustrated herein employs a steerable catheter, it should be appreciated that the present invention may also be applied to advantage in a range of applications employing non-steerable catheters. By way of a non-limiting example, the invention may be used to designate the location of vessel junctions or points of blockage during angioplastic stenting, or in any other application in which a point-of-interest is designated such as, but not limited to, the examples mentioned in the aforementioned PCT Publication No. WO00/16684.

It will be appreciated that the above descriptions are intended only to serve as examples, and that many other embodiments are possible within the spirit and the scope of the present invention.

What is claimed is:

1. A combined catheter and input device for use by a medical practitioner to designate points of interest reached by the device within the body of a patient, the device comprising:
(a) a catheter having a distal portion for insertion into the body and a proximal portion configured to be held by the practitioner;
(b) a location sensing system including at least one element associated with said distal portion of said catheter, said location sensing system determining a position of at least one point on said distal portion; and
(c) an input device mounted at said proximal portion and configured to be manually actuated to designate a current position of said at least one point as a point of interest.

2. The device of claim 1, wherein said catheter is a steerable catheter further including a steering mechanism manually operable from said proximal portion so as to steer said distal portion.
